(19) United States
(12) Patent Application Publication
(10) Pub. No.: US 2022/0061824 A1
(43) Pub. Date: Mar. 3, 2022

(54) Container for Sample Collection

(71) Applicant: ExSeed Health ApS, København S (DK)

(72) Inventors: Morten Gorm Ulsted, London (GB); Emil Andersen, København V (DK)

(21) Appl. No.: 17/309,885

(22) PCT Filed: Dec. 30, 2019
(86) PCT No.: PCT/EP2019/087161

§ 371 Date: Jun. 25, 2021

(30) Foreign Application Priority Data
Jan. 9, 2019 (DK) PA 2019 70011

(51) Int. Cl. A61B 10/00

(52) U.S. Cl. CPC A61B 10/0058; A61B 10/0096

(57) ABSTRACT

Disclosed is a container for collection of a biological sample, specifically semen, comprising a cup having an open top and an opposite, closed bottom, an accommodating space for receiving said sample and a depression defined by a side wall and a raised part of said bottom, a lid attachable to said top of said cup for closing in an assembled position of the container, wherein the lid comprises a pipetting element with a channel, so that when the lid and cup are attached in the assembled position of the container, the pipetting element extends into the depression of said cup, whereby said channel enables fluid communication between an outside environment of the container and said depression.

### CROSS-REFERENCE TO RELATED APPLICATIONS

This application is the U.S. National Phase of, and claims priority to, International Application No. PCT/EP2019/087161, filed 30 Dec. 2019, which designated the U.S. and which claims priority to Danish Application No. PA 2019 70011, filed 9 Jan. 2019.

### FIELD OF THE INVENTION

The invention relates to a container for sample collection, specifically a biological sample such as semen. The invention also relates to a method for collecting an aliquot of said sample, a method for detecting characteristics of said sample, a method for stirring said sample and a method for determining a weight of said sample.

### BACKGROUND OF THE INVENTION

In the traditional field of sample collection and testing, specifically biological samples such as semen, the sample is commonly delivered from the test subject directly to a one-time-use container.

The samples vary widely in volume between men from very small amounts (0.1 millilitres) to 10 millilitres (According to World Health Organization (WHO)). A low semen volume could indicate a low amount of sperm to fertilize an egg. An excess fluid volume could also mean the amount of sperm present is diluted. The sample must be analysed within one hour of collection.

A lab technician receives the container with the sample, and every time an aliquot of the sample must be taken out, a single use pipette is used to transfer the aliquot from the container to for example a testing area such as a microscope slide.

A new pipette is thereafter used to transfer the entire sample (after liquefaction) to a measuring tube to measure total volume of the sample.

It takes 15 to 30 minutes before semen liquefies. While semen is initially thick, its ability to liquefy, or turn to a watery consistency, helps sperm to move.

This procedure risks contamination of the sample by repeatedly opening the container and using a plurality of different instruments on the sample.

In recent years different home-testing kits have appeared on the market, enabling the test subject to avoid a trip to the doctor, by being able to test the quality of their semen in the comfort of their own home.

Here, different containers have been suggested for home testing. Even though the test is performed at home, it is still important to ensure that the sample is not contaminated and that the test subject is able to handle the sample properly.

Furthermore, the containers are often expensive to produce and thus the end-product will also be costly for the consumer, thereby restricting the market to certain users.

Thus, there is a need for a simple to produce and simple to use, versatile container, that may be used both by the medical professionals and the home user, whilst still providing a low risk of contamination.

### SUMMARY OF THE INVENTION

The following presents a simplified summary of the invention in order to provide a basic understanding of some aspects of the invention. This summary is not an extensive overview of the invention. It is not intended to identify critical elements of the invention or to delineate the scope of the invention. Its sole purpose is to present some concepts of the invention in a simplified form as a prelude to the more detailed description that is presented elsewhere herein.

On this background it may be an object of the invention to provide such a container and/or to alleviate, reduce or solve the problems and issues in the prior art.

Other objects of the invention may include providing a method for easy and simple collection of an aliquot and/or a method for easy and simple detecting of sample characteristics and/or a method for easy and simple handling of a sample and/or a method for easy and simple determination of the weight of a sample.

These and further objects may be met by the invention, which, according to a first aspect, relates to a container for collection of a biological sample, specifically semen, comprising

a cup having an open top and an opposite, closed bottom, an accommodating space for receiving said sample and a depression defined by a side wall and a raised part of said bottom,

a lid attachable to said top of said cup for closing in an assembled position of the container,

wherein the lid comprises a pipetting element with a channel, so that when the lid and cup are attached in the assembled position of the container, the pipetting element extends into the depression of said cup, whereby said channel enables fluid communication between an outside environment of the container and said depression.

Hereby a simple container is provided, that may be easily handled by both the home-user and the medical professional.

By providing the pipetting element in cooperation with the lid, the need for additional devices to perform a variety of test on/with the sample is avoided and thereby the risk of contamination of the sample is reduced.

Furthermore, the container according to the invention will be easy and economically advantageous to produce.

When the lid is arranged on the top of the cup, in the assembled position of the container, the accommodating space is enclosed by the lid and the cup.

In embodiments the lid comprises a body part, said pipetting element extending away from said body part.

When the container is in the assembled position, said body part is arranged on the top of the cup.

In embodiments the channel extends through the entire length of the pipetting element, preferably also through the body part of the lid.

In embodiments said pipetting element extends substantially along the entire length from the top of the cup towards the bottom of the cup in the assembled position of the container.

In embodiments said pipetting element extends substantially along the entire length of the side wall, from the top of the cup towards the bottom of the cup in the assembled position of the container.

With the development of the above described sample container the inventors of the invention have discovered, that it is important to have a simple to produce and handle container, that can accommodate for both small and larger biological samples, whilst still maintaining its functional properties with respect to sample testing.

This is especially important when the biological sample is semen, since the amount of semen that will be ejected during an ejaculation will vary widely between men and may contain between 0.1 and 10 milliliters (by way of comparison, note that a teaspoon is 5 ml and a tablespoon holds 15 ml).

Thus the container needs to allow for both small samples and larger samples, so it is possible to perform desired tests on the samples regardless of size.

The container according to the invention may solve this problem.

In embodiments the volume of a sample, that may be accommodated in the container is larger than or equal to 20 ml, preferably larger than or equal to 40 ml, more preferred larger than or equal to 55 ml.

In embodiments the maximum volume of a sample, that may be accommodated in the container is approximately 60 ml.

In the context of the specification, the term “pipetting element” may be understood as an element for transfer of a quantity, e.g. an aliquot.

In embodiments a part of the pipetting element extends into said sample, when the container is in the assembled position, so that when a sample is present in the container an aliquot of said sample is collected in the channel of said pipetting element, preferably by capillary action.

Hereby it is possible to collect the aliquot without having to open the container, since the channel in the pipetting element may be shaped so that said aliquot will flow into said channel without the assistance of additional equipment, and/or even in opposition to external forces like gravity.

The capillary action occurs because of intermolecular forces between the sample and the surrounding solid surfaces, in this case the pipetting element. Since the diameter of the channel is sufficiently small, then the combination of surface tension (which is caused by cohesion within the sample) and adhesive forces between the sample and pipetting element act to propel the liquid into the channel.

In embodiments the smallest diameter of the channel in the pipetting element is approximately 0.1-25 mm, preferably approximately 0.5-15 mm, more preferred approximately 0.75-5 mm.

In embodiments the diameter of the opening of the channel is approximately 1 mm.

In embodiments the aliquot is approximately 1-20 micro-litters, preferably approximately 5-15 micro-litters, more preferred approximately 10 micro-litters.

When the lid, and thus the pipetting element with the aliquot, is removed from the cup, the aliquot will remain in the channel due to the small size of the opening of said channel and the appertaining surface tension.

Hereby the invention provides an easy and simple way to collect an aliquot from the sample, regardless of sample size, and transfer it to a desired location such as a microscope slide for further analysis.

In embodiments the depression is an annular depression, so that when the lid is rotated on the cup in the assembled position of the container, said part of the pipetting element, that extends into said depression, is moved around the raised part of the bottom in said annular depression so as to be able to stir a sample present in the depression.

When the container is in the assembled position, the lid is arranged on the top of the cup. It is to be understood that the lid may be rotated whilst still being arranged on the cup. Hereby approximately the same distance from an end of the pipetting element to the bottom is maintained, while the lid is being rotated on the cup.

Hereby it is possible to use the pipetting element to stir the sample in the cup, thereby avoiding use of an additional element coming into contact with the sample and possible contaminating it.

Likewise the production cost is low, since only the container needs to be produced.

Furthermore, when the container is used for home testing, it may facilitate easy and uncomplicated handling of the sample for the user.

In embodiments the container comprises a measuring scale and/or a measurement element, preferably for measuring a pH value, and/or an enzyme, preferably a digestive enzyme, and/or a reagent.

In embodiments the measurement element is a test paper, specifically a pH indicator.

In embodiments a reagent may be added to estimate the pH value of the sample. Such a reagent may be Phenol Red, neutral red and/or bromothymol-blue.

In embodiments the measuring element is part of the lid and/or cup.

The measuring element may be molded as part of the container.

Hereby an easy way of determining characteristics of the sample, such as the pH value and/or volume and/or liquefaction, is provided by the invention.

Having the measurement element and/or enzyme already available in the container, the testing of the sample characteristics may be performed in a simple and easy manner. Even for the home user.

In embodiments the digestive enzyme may be coated on the cup, specifically on an inside surface of said cup.

In embodiments the digestive enzyme may be selected from the group consisting of: α-Chymotrypsin, Papain, Bromelain, α-Amylase or a combination thereof.

In embodiments the reagent may be selected from the group consisting of: Viability stain (Nigrosin/Eosin), Morphology (SpermBlue), DNA Fragmentation (GoldCyto DNA), Acrosome staining (eg. AcroScreen), Leukocyte stain (eg. LeucoScreen) or a combination thereof.

In embodiments a reagent may be added. Such a reagent may be Phenol Red, neutral red and/or bromothymol-blue.

The digestive enzyme may be sprayed on the inside of the cup, for example as a powder in a liquid. The cup may then be freeze-dried to set the enzyme.

Approximately 10% of all semen samples are too high in viscosity to be able to sample an aliquot. Thus it is necessary to add a digestive enzyme that breaks down the proteins in the sample, so it becomes lower in viscosity.

It is thus advantageous having the digestive enzyme already present in the container, when the sample is delivered therein.

Furthermore, as previously described, the pipetting element may be used to stir the sample and thereby mix the digestive enzyme with said sample.

In embodiments the pipetting element is tapered and/or conical.

In embodiments the pipetting element comprises a plane end surface.

In embodiments the pipetting element may be a truncated cone.

In embodiments a distance d between the pipetting element and a bottom of the depression and/or bottom of the cup is 0.1-20 mm, preferably 0.1-10 mm, more preferred 0.25-5 mm, when the container is in the assembled position.

In embodiments the distance d between the pipetting element and a bottom of the depression and/or bottom of the cup is approximately 0.5 mm.

In embodiments the distance d between the pipetting element and a bottom of the depression and/or bottom of the cup is approximately 3 mm.

In embodiments a distance between the plane end surface of the pipetting element and a bottom of the depression is 0.1-20 mm, preferably 0.1-10 mm, more preferred approximately 0.25-5 mm, when the container is in the assembled position.

Having a plane end surface and/or a truncated cone shape of the pipetting element ensures that the capillary action will occur even with very small amounts of the sample present in the cup, since only the tip of the pipetting element needs to penetrate the sample.

The cone shape of the pipetting element ensures that the sample will slide off the pipetting element, which is advantageous both during transfer of an aliquot, but also during determination of sample characteristics, where an aliquot of the sample must drip from the pipetting element to determine e.g. dripping frequency or a length of stringiness to determine a level of liquefaction.

In embodiments an incubation period of the sample, preferably in the container, must be performed prior to testing.

In embodiments the pipetting element is tapered away from the body of the lid, so that the diameter of said pipetting element becomes gradually smaller towards the bottom of the cup, when the container is in the assembled position.

In embodiments the pipetting element is shaped as a Zahn cup, which is advantageous when performing viscosity measurements.

In embodiments the pipetting element comprises a dent, which is advantageous when performing viscosity measurements.

In embodiments the raised part of the bottom extends towards the open top of the cup.

In embodiments the raised part of the bottom has a semi-spherical surface so that when a sample is transferred into the cup, said sample will glide from the raised part into said depression.

Thus an even distribution of the sample in the cup is ensured.

Additionally, the raised part of the bottom ensures, that even small samples will be gathered in the depression, where the pipetting element extends into and thus easily may come in to contact with.

In embodiments the pipetting element comprises a cap, specifically a deformable cap, so that said cap is able to create a vacuum above and/or in the channel, and selectively release said vacuum to draw up an aliquot of the sample present in the cup into the channel.

Hereby it is possible to move a larger aliquot from the sample.

In embodiments the deformable cap may be shaped as a pipette bulb.

In embodiments at least two lids, preferably a plurality of lids, are nestable and/or at least two cups, preferably a plurality of cups, are nestable.

It is to be understood that in the context of this specification the word “nestable” is to be understood as being “stacked within each other” or “stackable”.

When prior art containers in un-used form are packed and shipped, they take up a large amount of space due to the design of the containers. Furthermore, many of the containers have a design, where it is not possible to even stack the containers on top of each other.

By creating a container according to the invention, it is possible to maintain the functional properties of being able to handle and test varying sample sizes, whilst also being able to stack and pack the containers more closely together, since the lids and/or the cups are nestable, thus resulting in more economical packing and shipping.

The raised part of the bottom and/or the side walls of the cup and/or sides of the raised part are also shaped so that the cups are nestable, specifically closely nestable. Hereby the stacking of the cups is optimized.

In embodiments, the lid and/or the cup are/is manufactured from a material comprising at least 10%, at least 20%, at least 30%, at least 40%, at least 50%, at least 60%, at least 70%, at least 80%, at least 90% or at least 95% of a material or a combination of materials selected from the group consisting of silicone, silicone rubber, natural rubber, synthetic rubber, PTFE, polyethylene, polypropylene, HDPE, polystyrene, nylon and medico grade plastic.

In embodiments the medico grade plastic may be selected from the group consisting of PP_PCGH19, PP_PCGR40 or PP_PCGR25.

A material of the lid and/or cup may comprise additives and/or fillers, including coloring agents and/or softening agents.

In embodiments, the lid and/or the cup is/are manufactured by molding and/or 3D printing and/or cut from a base material.

The pipetting element and the body of the lid may be manufactured as one part.

The pipetting element may be molded directly onto the body of the lid.

In embodiments a part of the container, preferably the bottom of the cup, comprises a conductive material, so that when the container is arranged on a capacitive surface said surface may be activated.

In embodiments a substantial part, more preferred approximately the entire bottom of the cup comprises a conductive material.

In embodiments the entire bottom of the cup is substantially made of a conductive material.

The capacitive surface may be a touch screen, such as a smart phone screen.

Hereby it is possible to use the screen of an electronic device, specifically the 3D touch part of the screen, as a weight to weigh the container and the sample.

According to a second aspect the invention relates to a method for collecting an aliquot using said container as mentioned according to the first aspect of the invention, wherein said method comprises the steps of
arranging said sample in said accommodating space of the cup,
assembling said lid to said top part of said cup so as to form the assembled position of the container, wherein a part of the pipetting element extends into said sample, so that the aliquot of said sample is collected in the channel of said pipetting element,
removing said lid from the cup, thereby enabling transportation of said aliquot to a desired destination, such as a microscope slide.

In embodiments the aliquot of said sample is collected in the channel of said pipetting element by capillary action.

It is to be understood, that if the container is in the assembled position, so that the lid is arranged on the cup, then said lid must be removed prior to the step of arranging said sample in said accommodating space of the cup.

By using only the sample container for collecting said aliquot and easy way of sample collecting is provided. Here the use for additional means are avoided, and thereby the risk of contamination is reduced.

Furthermore, the handling for the user is simplified and more economically advantageous, since the container in itself is all that is needed to collect and/or transfer the aliquot.

According to a third aspect the invention relates to a method for detecting a characteristic of a sample using said container according to the first aspect of the invention, said method comprising the steps of
arranging said sample in said accommodating space of the cup,
assembling said lid to said top part of the cup so as to form the assembled position of the container, wherein a part of the pipetting element extends into said sample, so that an aliquot of the sample may be collected by said pipetting element,
moving said lid after a predetermined period so as to determine said characteristics of said sample.

In the context of this specification the term “moving” is to be understood as any movement that alters the physical relationship between lid and the cup and/or separates the lid from the cup, specifically a movement wherein the lid is raised to a higher position or level than the cup.

In embodiments the lid is lifted from the cup after a predetermined period so as to determine said characteristics of said sample.

When the lid is removed from the cup, the pipetting element being part of said lid, is also removed and thus also the aliquot that may be present in the pipetting element.

In embodiments the collecting an aliquot is performed according to the second aspect of the invention.

It is to be understood, that if the container is in the assembled position, so that the lid is arranged on the cup, then said lid must be removed prior to the step of arranging said sample in said accommodating space of the cup.

In embodiments the predetermined period of time is approximately 0-45 min, preferably approximately 10-35 min, more preferred approximately 15-30 min.

After the predetermined period of time the sample may be stirred and thereafter transferred.

In embodiments the sample may be stirred during the predetermined period of time.

When looking at characteristics of a biological sample, such as semen, some of the characteristics that are interesting to examine, but not limited to, are as follows:
drip sample (to determine viscosity)
drip frequency (to determine viscosity)
length of stringiness (to determine a level of liquefaction and/or viscosity)
pH value

By providing a method according to the third aspect of the invention as explained in the foregoing, an easy and simple way of investigating different characteristics of the biological sample is provided.

According to a fourth aspect the invention relates to a method for stirring a sample using said container according to the first aspect of the invention, said method comprising the steps of
arranging said sample in said accommodating space of the cup,
assembling the lid to the top part of said cup so as to form the assembled position of the container, wherein a part of the pipetting element extends into said sample,

so that when the lid is rotated on the cup in the assembled position of the container, the part of the pipetting element, that extends into said depression, is moved around in said depression so as to be able to stir the sample present in the accommodating space.

In embodiments the depression is an annular depression.

In embodiments when the lid is rotated in the assembled position of the container, said part of the pipetting element, that is extends into said depression, is rotated around in said annular depression, specifically moved around a raised part of the bottom in said annular depression, so as to stir a sample present in the accommodating space.

In the context of this specification the term “stir” is to be understood as the action of moving, or causing to move at least slightly, or mixing, or blending, or a combination of those, or likewise movement of the sample.

According to a fifth aspect the invention relates to a method for determining the weight of a cup as according to the first aspect of the invention, specifically a cup comprising a biological sample, said method comprising the steps of
arranging said cup on a capacitive surface of an electronic device, preferably a smartphone screen,
measuring the force that the cup exerts on said capacitive surface so as to be able to determine the weight of the cup.

In embodiments the invention relates to a method for determining the weight of a cup as according to the first aspect of the invention, specifically a cup comprising a biological sample, said method comprising the steps of
arranging said cup with sample on a capacitive surface of an electronic device, preferably a smartphone screen,
measuring the force that the cup and sample exerts on said capacitive surface so as to be able to determine the weight of the sample.

By arranging the cup or the entire container with the sample on the screen of a smartphone, the container will exert a force on the screen, so that the smartphone may measure said force and from this calculate the weight of the container. Since the weight of the container is known, it is possible to determine the weight of the sample alone.

This will also allow a determination of the volume of the sample based on density and weight of the sperm.

### SUMMARY OF THE DRAWINGS

The above and/or additional objects, features and advantages of the invention will be further elucidated by the following illustrative and non-limiting detailed description of embodiments of the invention, with reference to the appended drawings, wherein:

FIG. 1a shows a cross-sectional view of a lid of an embodiment of a container according to the first aspect of the invention.

FIG. 1b shows a cross-sectional view of a cup of an embodiment of a container according to the first aspect of the invention.

FIG. 2 shows a perspective cross-sectional view of a container according to the invention in an assembled position.

FIGS. 3a-b shows different perspective views of a lid according to an embodiment of the invention.

FIG. 3c shows a perspective view of the cup according to an embodiment of the invention.

FIG. 4a-d shows different perspective view and cross-sectional views of a plurality stacked lids and cups according to an embodiment of the invention.

### DETAILED DESCRIPTION OF THE INVENTION

In the following, embodiments of the invention will be described in further detail. Each specific variation of the features can be applied to other embodiments of the invention unless specifically stated otherwise. Note that for illustrative purposes the dimensions, especially thicknesses, of the different elements shown may be exaggerated.

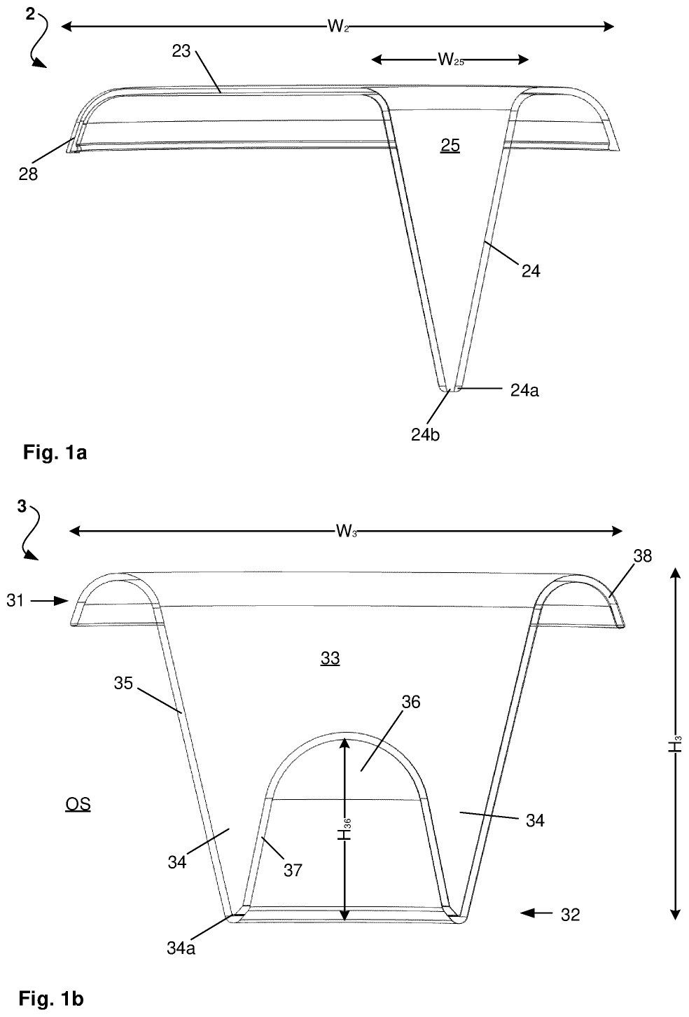

Turning first to FIG. 1a which shows a lid 2 according to an embodiment of the invention and FIG. 1b which shows a cup 3 according to an embodiment of the invention.

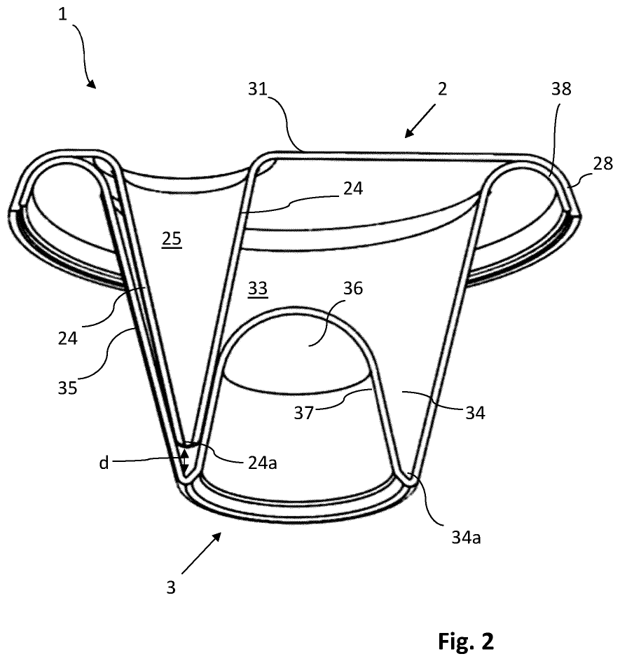

In an embodiment as shown on FIG. 2 a container 1 for collection of a biological sample, specifically semen, is shown. The container 1 comprises the lid 2 and the cup 3. The container 1 is shown in the assembled position.

The cup 3 has an open top 31 and an opposite, closed bottom 32, and a circumferential side wall 35 extending between the top 31 and the bottom 32.

When the container 1 is in the assembled position, the lid 2 is arranged on the top 31 of the cup 3. The lid 2 is thus attachable to said cup 3 for closing in the assembled position of the container 1.

The cup 3 further comprises an accommodating space 33 for receiving the sample (not shown).

The accommodating space 33 is to be understood as the free volume that the cup 3 may contain. The accommodating space may be closed off by the lid 2, when the container 1 is in the assembled position as shown on FIG. 2.

The cup 3 further comprises an annular depression 34 defined by the circumferential side wall 35 and a raised part 36 of said bottom 32.

The raised part 36 is centrally located, and extends from the bottom 32 towards the open top 31.

The raised part 31 has a semi-spherical surface towards the open top 31, so that when a sample is transferred into the cup, said sample will glide from the raised part 31 into said depression 34.

The depression 34 has a substantial conical shape to match the shape of a pipetting element 24 of the lid 2, as shown on FIG. 2.

The bottom of the depression 34a has a truncated cone shape, with a slanted end surface, so as to obtain a better distribution of small amounts of sample. This is advantageous e.g. during volume determination and/or collection of an aliquot.

The depression may have any shape that corresponds with the functionalities described in this specification.

The raised part 36 is hollow.

As seen on e.g. FIG. 1b, the circumferential side wall 37 functions both as a side wall of the raised part 36 and as a side wall of the depression 34.

The hollow raised part 36, the angle of the side wall 37 and the angle of the side wall 35 ensures that at least two cups are nestable.

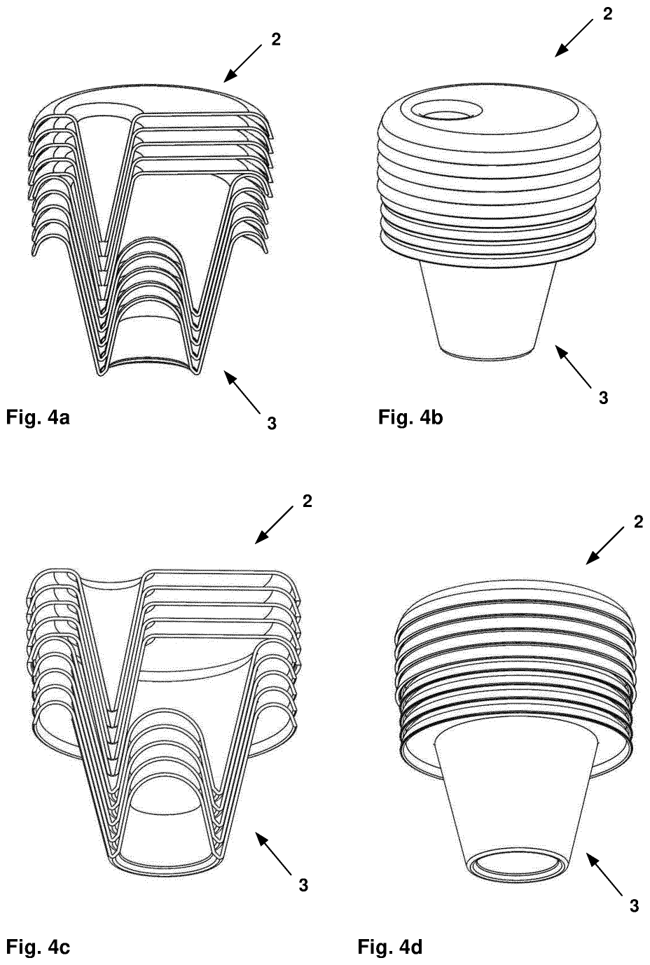

As shown on FIGS. 4a-d a plurality of cups are stacked within and/or on each other, so as to provide optimized stacking/packing.

The cup 3 further comprises a circumferential flange 38, that extends on the outside OS of the cup 3 from the top side 31 towards the bottom 32. The flange 38 is circumferential and forms together with a part of the side wall 35 (present at the top 31 of the cup 3) a semi-spherical shape, so as to facilitate easy handling of the cup and stacking.

The flange 38 is shaped so as to mate with a lid-flange 28 (as seen on FIG. 1a), so that the lid 2 may be releasably attached to the cup 3 so as to form the assembled position of the container 1.

In embodiments the height H₃ of the cup 3 is 45-55 mm, preferably 48-53 mm, more preferred approximately 51 mm.

In embodiments the widest/maximum width W₃ of the cup 3 (measured at the top 31) is 65-95 mm, preferably 75-85 mm, more preferred approximately 77 mm.

In embodiments the height H₃₆ of the hollow part of the raised part 36 (extending from the bottom 32 of the cup 3 and to an uppermost point towards the top 31 of the raised part 36) is 20-40 mm, preferably 25-35 mm, more preferred approximately 30 mm.

Turning to FIG. 1a where the lid 2 is shown.

The lid 2 is releasably attachable to the top 31 of said cup 3 for closing in an assembled position of the container 1 as shown on FIG. 2.

When the container 1 is in the assembled position, said body part 23 is arranged on the top of the cup 3.

The lid 2 comprises a body part 23 and a pipetting element 24. The pipetting element 24 extends out and away from the body part 23.

The pipetting element 24 comprises a channel 25, that extends through the entire length of the pipetting element 24.

The pipetting element 24 is shaped as a truncated cone, thus it has s cone shape lacking an apex and terminating in a plane end surface 24a.

The plane end surface 24a is approximately parallel with a plane of a surface that the cup may be arranged on.

When the container 1 is in the assembled position as shown on FIG. 2, the distance d between the pipetting element 24 and the bottom of the depression 34a is approximately 3 mm.

When the lid 2 and cup 3 are in the assembled position of the container 1, the pipetting element 24 extends into the depression 34 of said cup, whereby the channel 25 enables fluid communication between an outside environment OS of the container 1 and said depression 34.

Hereby it is possible to arrange a sample (not shown) in the accommodating space 33 of the cup 3, and thus in the depression 34, assemble the lid 2 to the top part 31 of the cup 3, so as to form the assembled position of the container 1, wherein a part of the pipetting element 24 will extend into the sample, so that an aliquot of the sample will be collected in the channel 25 of the pipetting element 24 by capillary action. When the lid 2 is subsequently removed from the cup 3, the aliquot is automatically transported in the channel 25 of the pipetting element 24. The aliquot may hereafter be transported to any desired destination, often a microscope slide. During transportation the small size of the opening 24b of the channel 25 and the appertaining surface tension ensures that the aliquot remains in the channel. When the pipetting element and aliquot comes into contact with another surface, such as the microscope slide, the surface tension is disrupted, and the aliquot will be transferred to said slide.

In one embodiment the diameter of the opening 24b of the channel 25 is 0.1-5 mm, preferably 0.-2.5 mm, more preferred approximately 1 mm.

The lid 2 may also be lifted from the cup 3 after a predetermined period of time so as to determine a characteristic of a sample present in the cup 3.

The pipetting element 24 extends substantially in the entire length, from the top 31 of the cup 3 towards the bottom 32 of the cup 3 in the assembled position of the container 1.

When in the assembled position the pipetting element 24 extends substantially in the entire length of the side wall 35, from the top of the cup towards the bottom of the cup.

The pipetting element 24 is positioned adjacent to the side wall 35 in the assembled position of the container 1.

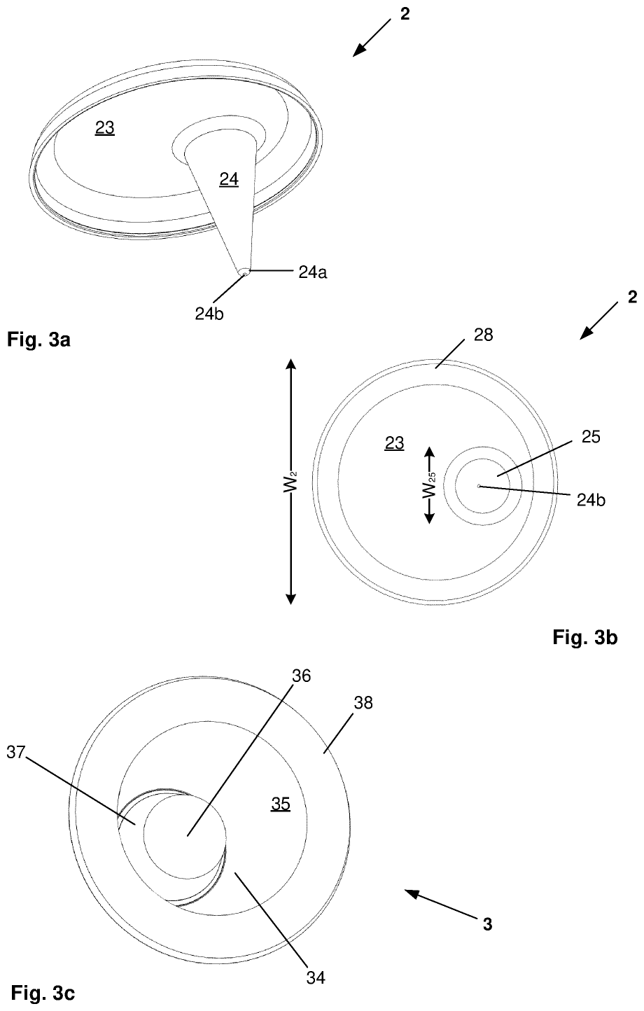

Turning to FIGS. 3a and 3b, it is shown that the pipetting element 24 is arranged off center in relation to the body part 23.

Thus it is possible for the pipetting element 24 to, in the depression 34, move around and/or rotate around the raised part 36.

Hereby when the lid 2 is rotated e.g. by a user on the cup 3 in the assembled position of the container 1, the part of the pipetting element 24, that extends into said depression 34, and thus into the sample, is moved around in said annular depression, so the sample present in the accommodating space is stirred.

The maximum width W₂ of the lid is 70-90 mm, preferably 75-85 mm, more preferred approximately 79 mm.

The maximum width W₂₅ of the channel is 15-35 mm, preferably 20-30 mm, more preferred approximately 25 mm.

The channel 25 is widest at the opening in the body 23 of the lid.

The channel 25 has a cone shape and tapers towards the plane end surface 25a of the pipetting element 24.

The minimum width of the channel 24 is the opening 24b at the end of the pipetting element 24.

The lid-flange 28 is shaped so as to mate with the flange 38 (as seen on FIG. 1b), so that the lid 2 may be releasably attached to the cup 3 so as to form the assembled position of the container 1.

Turning to FIGS. 4a-d the shape of the lid 2 ensures that at least two of lids 2 are nestable as shown on the figures, where a plurality of lids are stacked.

Additionally, the inventive shape of the container 1 allows for both a plurality of lids 2 and a plurality of cups 3 to be stacked, whereby packing is optimized.

Although some embodiments have been described and shown in detail, the invention is not restricted to them, but may also be embodied in other ways within the scope of the subject matter defined in the following claims. It is to be understood that other embodiments may be utilised and structural and functional modifications may be made without departing from the scope of the invention.

In device claims enumerating several means, several of these means can be embodied by one and the same item of hardware. The mere fact that certain measures are recited in mutually different dependent claims or described in different embodiments does not indicate that a combination of these measures cannot be used to advantage.

It should be emphasized that the term “comprises/comprising” when used in this specification is taken to specify the presence of stated features, integers, steps, or components but does not preclude the presence or addition of one or more other features, integers, steps, components, or groups thereof.

1. A container for collection of a biological sample, specifically semen, comprising:
a cup having an open top and an opposite closed bottom, an accommodating space for receiving said sample and a depression defined by a side wall and a raised part of said bottom, and
a lid 2 attachable to said top of said cup for closing in an assembled position of the container,
wherein the lid comprises a pipetting element with a channel, so that when the lid and cup are attached in the assembled position of the container, the pipetting element extends into the depression of said cup, whereby said channel enables fluid communication between an outside environment of the container and said depression.

2. A container according to claim 1, wherein a part of the pipetting element extends into said sample, when the container is in the assembled position, so that when a sample is present in the container an aliquot of said sample is collected in the channel of said pipetting element.

3. A container according to claim 2, wherein the depression is an annular depression, so that when the lid is rotated on the cup in the assembled position of the container, said part of the pipetting element that extends into said depression is moved around the raised part of the bottom in said annular depression so as to be able to stir the sample present in the depression.

4. A container according to claim 1, further comprising a measuring scale or a measurement element, for measuring at least one of a pH value an enzyme, and a reagent.

5. A container according to claim 1, wherein the pipetting element is at least one of tapered and conical.

6. A container according to claim 1, wherein at least two lids are nestable and at least two cups, are nestable.

7. A container according to claim 1, wherein a distance between the pipetting element and a bottom of the depression or a bottom of the cup is 0.1-20 mm when the container is in the assembled position.

8. A container according to claim 1, wherein a part of the container comprises a conductive material so that when the cup is arranged on a capacitive surface said surface may be activated.

9. A method for collecting an aliquot using said container as claimed in claim 1, wherein said method comprises the steps of;
arranging said sample in said accommodating space of the cup,
assembling said lid to said top part of said cup so as to form the assembled position of the container, wherein a part of the pipetting element extends into said sample so that the aliquot of said sample is collected in the channel of said pipetting element, and
removing said lid from the cup, thereby enabling transportation of said aliquot to a desired destination.

10. A method for detecting a characteristic of a sample using said container 1 as claimed in claim 1, said method comprising the steps of
arranging said sample in said accommodating space of the cup,
assembling said lid to said top part of the cup so as to form the assembled position of the container, wherein a part of the pipetting element extends into said sample so that an aliquot of the sample may be collected by said pipetting element, and
moving said lid after a predetermined period so as to determine said characteristics of said sample.

11. A method for stirring a sample using said container as claimed in claim 11, said method comprising the steps of:
arranging said sample in the depression of the cup, and
assembling the lid to the top part of said cup so as to form the assembled position of the container, wherein a part of the pipetting element extends into said sample present in the depression so that when the lid is rotated on the cup in the assembled position of the container, the part of the pipetting element that extends into said depression is moved around in said depression so as to be able to stir the sample present in the cup.

12. A method for determining the weight of a cup as mentioned in claim 8, specifically a cup comprising a biological sample, said method comprising the steps of:
arranging said cup on a capacitive surface of an electronic device, and
measuring the force that the cup exerts on said capacitive surface so as to be able to determine the weight of the cup.

13. A container according to claim 2, wherein said sample is collected in the channel of said pipetting element by capillary action.

14. A container according to claim 1, further comprising a measuring scale or a measurement element for measuring a pH value, an enzyme, and a reagent.

15. A container according to claim 4, wherein the enzyme is a digestive enzyme.

16. A container according to claim 1, wherein at least two lids are nestable or at least two cups are nestable.

17. A container according to claim 1, wherein a distance between the pipetting element and a bottom of the depression or a bottom of the cup is 0.1-10 mm when the container is in the assembled position.

18. A container according to claim 1, wherein a distance between the pipetting element and a bottom of the depression or a bottom of the cup is 0.25-5 mm when the container is in the assembled position.

19. A container according to claim 8, wherein the bottom of the cup of the container comprises the conductive material, so that when the cup is arranged on said capacitive surface said surface may be activated.

20. A method as claimed in claim 12, wherein said capacitive surface is a smartphone screen.
